(19) United States
(12) Patent Application Publication
(10) Pub. No.: US 2011/0313416 A1
(43) Pub. Date: Dec. 22, 2011

(54) ELECTROHYDAULIC PROSTATE TISSUE TREATMENT PROBE

(76) Inventor: Qijun Jiang, Liaoning (CN)

(21) Appl. No.: 13/138,506

(22) PCT Filed: Jan. 15, 2010
(86) PCT No.: PCT/CN2010/070195

§ 371 Date: Aug. 24, 2011

(30) Foreign Application Priority Data
Feb. 24, 2009 (CN) 20091010438.7

(51) Int. Cl. A61B 18/14

(52) U.S. Cl. 606/41

(57) ABSTRACT

An electrohydraulic prostate tissue treatment probe comprises a probe pipe body (10). A drainage channel (2-1), a water-injection channel (1-1), a gas-injection channel (3-1) and a cathode channel (5) are arranged in the probe pipe body (10) along the axial direction. The front end of the probe pipe body (10) is a guiding head (9), behind which a positioning balloon (8) is arranged. An external cathode electrode (7) is wound tightly around the probe pipe body (10) on the rear side of the positioning balloon (8). The leading-out end of the external cathode electrode (7) is connected with a cathode plug (A) through the cathode channel (5). An external anode electrode (6) is arranged between the positioning balloon (8) and the cathode electrode (7), and is wound around the probe pipe body (10) by at least one circle. The external anode electrode (6) is kept at a distance from the cathode electrode (7). The leading-out end of the external anode electrode (6) is connected with an anode plug (B) through the drainage channel (2-1).

### BACKGROUND OF THE PRESENT INVENTION

1. Field of Invention

The present invention relates to treatment probe, and more particularly to an electrohydraulic prostate tissue treatment probe which is the preferred treatment probe of medical institutions to treat benign prostate hyperplasia by electrolysis.

2. Description of Related Arts

Prostate hyperplasia or prostate enlargement is a common disease found in older men which includes the major symptom of progressive difficulties in urination. Early symptoms include frequent or urgent urination and nocturia. When bladder blockage obstruction is developed, the symptoms include urinary retention, weak or interrupted stream of urine and sense of incomplete voiding or hesitancy. When the detrusor muscle of bladder loses its compensatory function, serious or life-threatening complications may develop which includes overflow incontinence, hydronephrosis and renal failure. Acute diseases which include abdomen swelling, urinary tract stone or infection, bladder bleeding and urinary retention may also be developed. There is no existing inhibitory procedure to prevent prostate enlargement and no medications which is capable of preventing bladder obstruction or renal retention. At present, TURP (Transurethral Resection of the Prostate) is the gold standard for treating an enlarged prostate at the worldwide level. In recent years, many hospitals which are graded at or above city level in our country have developed to deploy TURP. However, since the operation of the procedure is very difficult, there is only a few number of professional surgeons who are skillful to operate the procedure in different provinces or districts of our country. In particularly, old aged patients who are weak and unfit for anesthesia are not fit to have this operation and can only relied on leak management or urinary catheterization. Accordingly, Professor Qi Jun Jiang who is an academic in urology of our country has successfully developed an “electrolytic treatment probe for prostate”, applied for an inventive patent application on 19 Jan. 1993 and obtained the patent rights thereof in which the patent number is ZL93110056.9 and the disclosure is a method of treatment of prostate enlargement through urinary tract intervention, which is based on 15-year clinical trials with more than 300 practitioners and their feedback and has more than 100 successful cases. In the clinical trials, the successful rate is more than 87% with no reported cases of dead during treatment and within the treatment period. This method is therefore the safest treatment method for treating prostate enlargement in the country. Nevertheless, through the research and pathological studies in vivo and in vitro of more than one hundred cases of prostate enlargement, the treatment probe for treating prostate enlargement has the following identified drawbacks which require further improvement. First, the range of treatment parameter of liquefied tissue is narrow of which the maximum radius is 10 mm, therefore patients with relatively high level of prostate enlargement may experience blockage again in two years after treatment. Second, in the later stage of treatment, the amount of tissue debris is significant and the drainage channel may be blocked easily, therefore the anode inside the drainage channel is surrounded by the gaseous membrane and current interruption during treatment may occur. Third, the anode is made of expensive platinum-rhodium alloy wire, sophisticated sterile procedure is required for repeated use or cross infection may occur.

### SUMMARY OF THE PRESENT INVENTION

The invention is targeted at solving the existing problem to provide an electrohydraulic treatment probe for treating prostate tissue which provides a large treatment diameter, an enhanced recovery process for treated tissue, a significantly enhanced treatment effect without relapse, a stable treatment current without affecting the treatment procedure even the drainage channel is blocked while cross infection is prevented and the cost is lowered.

Another object of the present invention is to provide a method of treating prostate enlargement with a non-operational procedure through electrohydraulic treatment which is introduced through urethra of which the surface of urethra is anesthetized, thereby the method can be easily performed in a clinic with simple operation procedure and is acceptable to the patient with low level of undesirable effect, wherein the method is particularly suitable for use in older aged and high risk objects who is not fit for operation and for use in object with early stage of prostate enlargement, and the method is suitable and safe for use in medium or small sized hospital to develop an effective and practical way of treatment.

The present invention is realized by means of the followings:

According to the present invention, the foregoing and other objects and advantages, the present invention includes an electrohydraulic prostate tissue treatment probe, comprising: a probe pipe body (10), wherein the probe pipe body (10) has a drainage channel (2-1), a water-injection channel (1-1), a gas-injection channel (3-1) and a cathode channel (5) longitudinally provided in the probe pipe body (10) along an axial direction; a guiding head (9) provided at a front end of the probe pipe body (10); a positioning balloon (8) provided behind the guiding head (9); and an external cathode electrode (6) tightly surrounding the positioning balloon (8) at a rear position and having an extended end connected to a cathode plug (A) through the cathode channel (5), wherein the treatment probe is characteristic in that: the external anode electrode (6) is positioned between the positioning balloon (8) and the cathode electrode (7) and is surrounding the positioning balloon (8) for at least one cycle such that a predetermined distance between the anode electrode (6) and the cathode electrode (7) is maintained and the extended end is connected to the anode plug (B) through the drainage channel (2-1).

The distance between the anode electrode (6) and the cathode electrode (7) is 3-5 mm.

The anode electrode is a platinum-rhodium alloy wire which has a diameter of 0.3 mm.

The probe pipe body (10) has a wire channel (4) longitudinally provided along the axial direction within an effective length of the probe pipe body (10) and a flexible wire extended between two ends of the wire channel (4) to connect with the probe pipe body to secure into position.

The cathode electrode (7) is spacedly provided along the axial direction defining a plurality of cathode sections which has an interval of 5 mm, wherein one of the cathode section has a length of 5-10 mm while other cathode sections each has a length of 5 mm.

Through extensive experiments and research studies in vivo and in vitro, the results indicate that the anode electrode which is positioned externally between the positioning balloon and the cathode electrode, is complementary to the external cathode electrode at a position near to a rear portion of the positioning balloon, thereby the treatment is enhanced, the range of treatment diameter is increased, the recovery period of patient is shortened or the single treatment time is shortened. Accordingly, this poses an important and significant effect on the safety and reliability level to obtain the best result. Conversely, if the external anode is positioned in front of the positioning balloon on the guiding head, the path is lengthened and the current cannot be adjusted to increase easily while the range of treatment diameter is not increased. In addition, if the condition of emptied bladder is not or cannot be discovered on a timely basis during treatment, the anode electrode which is in contact with the wall of the bladder will lead to damage to the wall of bladder or even destruction to the wall of bladder, which is not desirable or preferable.

The present invention has the following advantageous effect:

Compared to conventional technology, the present invention includes an anode electrode positioned externally between the positioning balloon and the cathode electrode near the positioning balloon and feature of an additional wiring element which prevents stretching and distortion of silica pipe. In addition, the circumferential area of the electrohydraulic prostate tissue treatment probe is tested and well-defined through clinical trails, wherein the liquefied dead tissue is in the form of columnar shape. When the treatment power is increased, the process of liquefying dead tissue occurs near the electrode area and spread outwardly gradually and the maximum radius of 15 mm or above can be reached. A size of the treatment probe is selected based on a length of the urethra through the prostate, then the process of treatment will be safe and reliable without causing any damage to the non-targeted organs. The liquefied dead tissue will be shredded off and drained out with washing, and do not have any contact with the electrode. All the liquefied dead tissue is removed completely within one month after treatment and the occurrence of relapse is not likely. The basal wound surface is healed through mucosal lining of urethra, thereby scar tissue and fiber is not likely to grow and accumulate. Even when the drainage pipe is blocked by the shredding tissue, the treatment current can still be maintained under the preset condition without affecting the process of the procedure. On the other hand, the present invention can significantly lower the cost of materials of the probe in which the material requirement of platinum-rhodium alloy electrode is only 5% of the conventional probe, while the probe of the present invention can effectively eliminate cross infection, is acceptable to the patient with less side effect and suitable for implementation in all kinds of hospital for treating prostate enlargement, especially for those old aged and high risk patients who is not fit for having operation procedure and being anesthetized.

Still further objects and advantages will become apparent from a consideration of the ensuing description and drawings.

These and other objectives, features, and advantages of the present invention will become apparent from the following detailed description, the accompanying drawings, and the appended claims.

### BRIEF DESCRIPTION OF THE DRAWINGS

FIG. 1 is a structural illustration of a treatment probe according to a preferred embodiment of the present invention.

FIG. 2 is an illustration of an A-A cross-sectional view of the treatment probe according to the above preferred embodiment of the present invention.

In the figures: 1: water-injection opening; 2: drainage opening; 3: gas-injection valve; 4: wiring channel; 5: cathode channel; 6: anode electrode; 7: cathode electrode; 8: positioning gas vesicle; 9: guiding head; 10: probe body; 11: drainage inlet; 12: gas exit; 13: fluid exit; 1-1: water-injection channel; 2-1: drainage channel; 3-1: gas-injection channel; A: cathode plug; B: anode plug.

### DETAILED DESCRIPTION OF THE PREFERRED EMBODIMENT

The preferred embodiment of the present invention is further described in details with the following drawings and is not intended to be limiting.

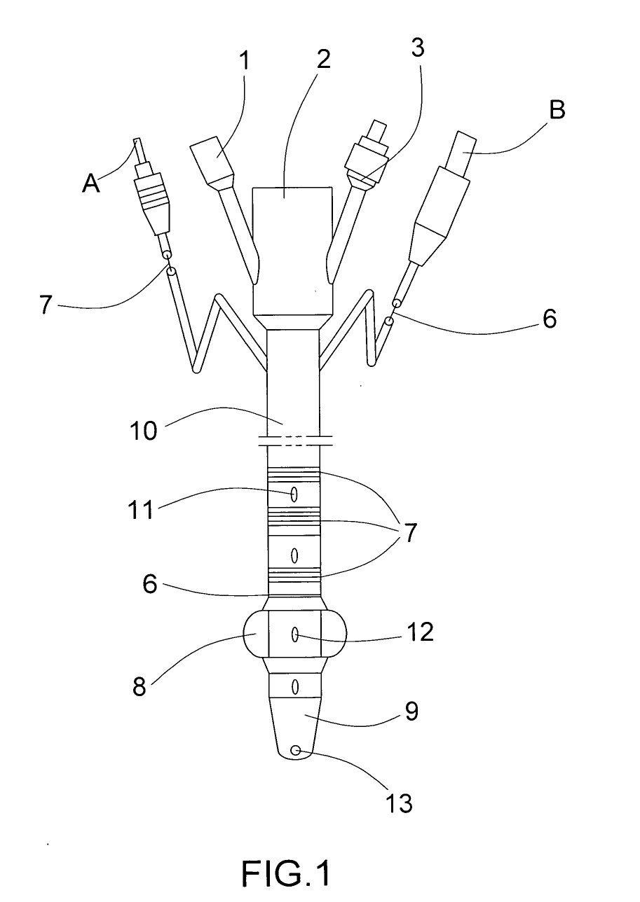

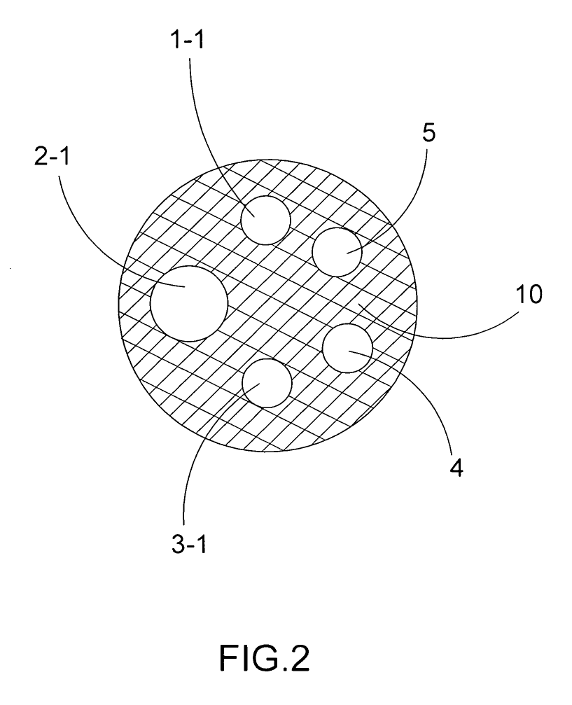

Referring to FIG. 1 of the drawings, a structural illustration of a treatment probe according to a preferred embodiment of the present invention is illustrated. The probe pipe body 10 is made of medical use silica which has a front end defining a guiding head 9; a fluid exit 13 and a drainage inlet 11 provided thereon; a positioning balloon 8 provided adjacent to the drainage inlet 11 away from the front end; a gas exit 12 provided on a side wall of the probe pipe body 10 at which the side wall is surrounded by the positioning balloon 8; a cathode plug A, a water-injection opening 1, a drainage opening 2, a one-way gas-injection valve 3 and an anode plug B, each channeled through its own channel, provided at a rear end of the probe pipe body 10 respectively, wherein the cathode plug A has an opposite end connected to a cathode electrode which is positioned externally through a medical use aluminum alloy wire of smaller than 0.3 mm provided in the cathode channel 5, the drainage exit 2 is channeled to the drainage inlet 11 through the drainage channel 2-1, the water-injection opening 1 is channeled through the fluid outlet 13 through the water-injection channel 1-1, the gas-injection valve 3 is channeled to the gas exit 12 through the gas-injection channel 3-1, the anode plug B is connected to the anode electrode 6, which is positioned externally between the positioning balloon 8 and the cathode electrode 7, through a platinum-rhodium alloy wire of 0.3 mm which passes through the drainage channel 2-1. The anode electrode 6 is a coil having 1-3 turns and is positioned near a rear portion of the positioning balloon 8 and has a distance of 3-5 mm from the cathode electrode 7. The cathode electrode 7 is spacedly and discretely provided on the probe pipe body 10 along the axial direction defining a plurality sections of cathode electrode 7, wherein each two cathode electrode 7 has an interval distance of 5 mm and each section has a length of 5 mm, wherein each section is capable of having a different length with respect to each other and one of the section can has a length of 5-10 mm. The length is a distance of one turn of one section of the electrode which surround the probe pipe body. Each section of the electrode has one drainage inlet which is opened through a side wall of the probe pipe body 10 to channel through the drainage channel 2-1. The probe pipe body 10 has an effective length along the axial direction within which a wiring channel 4 is provided and arranged for preventing stretching and distortion of the wiring element. A positioning wiring element or arrangement is provided in and extended between two ends of the wiring channel 4 to connected to the probe pipe body 10 to secure into position. As shown in FIG. 2 of the drawings, this can effectively prevent stretching and distortion and hence prevent messing up the position of the electrodes such that an accurate positioning of the electrodes for the treatment probe is provided.

Application embodiment: Mr. Zhang, male, aged 88, diagnosed as prostate enlargement with urine retention. Based on the patient's length of urethra of prostate portion of 58 mm, a 50 mm prostate probe is selected. The electric charged applied is 400 Coulomb (based on 30 mm prostate probe which utilizes 200 coulomb, the electric charge is increased by 50 coulomb for each increase of 5 mm in length of electrode). The probe is inserted to position into the bladder by catheterization. The positioning balloon 8 is filled with 10 ml water. The external pulling probe is blocked and stop pulling. When there is no pulling force, the treatment probe is secure into position. When all the connecting elements are ready, the remaining urine in the bladder is emptied out and the drainage pipe is closed. Start infusing 200 ml physiological saline into the bladder, open drainage pipe which is connected to the urine collection bag which is positioned at a distance of 100-200 mm from the pubis (public bone) so as to maintain a predetermined quantity of washing fluid in the bladder while allowing smooth passage, and setting and controlling the drainage rate at 1000 ml/hour. Turn on the power to start treatment. Initially, apply a current of 30 milliampere. When the electric charge is accumulated to reach 50 coulomb, gradually increase the treatment current to 80 milliampere (±10 milliphere) according to the tolerance of the patient until the required treatment electric charge is reached. During treatment, maintain a drainage flow rate of 1000 ml/hour for washing. After the treatment electric charge is reached, keep infusing 200 ml physiological saline to the bladder, remove the fluid inside the positioning balloon and pull out the treatment probe. Then, immediately place a F16 catheter and continue catheterization for 24 hours to 2 weeks.

A clinical trial of 12 patients, aged between 57-88, weight of prostate between 35-67 g, is conducted and the above belongs to one of the cases of the clinical trial. All patients suffered from urine retention.

Amongst all the patients, only superficial anesthetization of urethra is required to having treatment under a safe and stable condition. No abnormal or undesirable effect is observed. During the treatment process, the current is steady and the variation is less than 5 milliampere. No power cut-off alarm phenomenon for interrupted drainage occurs. Between 24 hours to 2 weeks after treatment, all patients can complete control over urination. The urine flow rate test is conducted after 3 weeks and the results of which shows that the maximum flow rate has an average which is greater than 25 ml/second. All cases are observed for 1.5 to 2 years and all patients are recovered to have complete control of urination. No relapse of urine retention is observed.

Further in vivo and in vitro tests are conducted and the test results are supportive to the results of the clinical trial. The testing object is muscle tissue of the same type of animal to which the same level of electric charge is applied as treatment. Comparative testing between the treatment probe of the present invention and the convention probe is conducted to determine the range and effect of tissue liquefaction. The test results show that tissue liquefaction range at the apex of prostate is 17-20 mm when the treatment probe of the present invention is used while that of the convention probe is smaller than or equal to 10 mm. Cytoscopic examination of bladder outlet of the patients show that the results are the same as the test results of the in vivo and in vitro tests.

### CONCLUSION

1. Through in vivo and in vitro testing and clinical trial, the treatment probe of the present invention has a significant and obvious increased range of liquefaction of prostate tissue when compared to conventional probe. The treatment probe of the present invention can completely liquefy the dead tissue without causing damage to non-treated organ and relapse is not likely to occur after treatment;

2. The current fluctuation is small during the treatment process. The treatment process is not stopped or interrupted owing to drainage interruption. The drawbacks of great current fluctuation and open circuit which are common to conventional probe are effectively eliminated;

3. After treatment, the time period of patient to recover and resume control over urination is shortened by 1-2 weeks, and the maximum flow rate and the treatment effect are significantly enhanced.

4. Since the electrohydraulic prostate tissue treatment probe is used to liquefy and kill the prostate tissue around the probe during treatment to form a columnar pattern, when the treatment power is increased, the treatment area is spread outwardly from the area near the electrode until the required treatment power is reached and the maximum treatment range is reached. Though dead tissue is also induced in the anode area around the bladder outlet, the dissolving effect of fluid inside the bladder together with the cathode which is circumferentially surround by the liquefied and dead tissue can effective prevent the tissues from contacting the electrodes. The treatment probe can be removed immediately after treatment and is not required to be placed inside the body, therefore the time of recovery is shortened;

5. This is the preferred method, which is safe and reliable, for older aged and high risk patients who is not fit for having operation. The recovery time is short;

6. This method can lower the economic burden of patient, can significantly reduce the cost of treatment. Compared to having an operation, about ⅔ of the cost of having an operation can be saved. No hospitalization is required after treatment, therefore the economical burden for the patient is lowered while the resources of the hospital is saved.

One skilled in the art will understand that the embodiment of the present invention as shown in the drawings and described above is exemplary only and not intended to be limiting.

It will thus be seen that the objects of the present invention have been fully and effectively accomplished. It embodiments have been shown and described for the purposes of illustrating the functional and structural principles of the present invention and is subject to change without departure from such principles. Therefore, this invention includes all modifications encompassed within the spirit and scope of the following claims.

What is claimed is:

1. An electrohydraulic prostate tissue treatment probe, comprising: a probe pipe body (10), wherein the probe pipe body (10) has a drainage channel (2-1), a water-injection channel (1-1), a gas-injection channel (3-1) and a cathode channel (5) longitudinally provided in said probe pipe body (10) along an axial direction; a guiding head (9) provided at a front end of said probe pipe body (10); a positioning balloon (8) provided behind said guiding head (9); and an external cathode electrode (6) tightly surrounding said positioning balloon (8) at a rear position and having an extended end connected to a cathode plug (A) through said cathode channel (5), wherein said treatment probe is characteristic in that: said external anode electrode (6) is positioned between said positioning balloon (8) and said cathode electrode (7) and is surrounding said positioning balloon (8) for at least one cycle such that a predetermined distance between said anode electrode (6) and said cathode electrode (7) is maintained and said extended end is connected to said anode plug (B) through said drainage channel (2-1).

2. The electrohydraulic prostate tissue treatment probe, as recited in claim 1, wherein said distance between said anode electrode (6) and said cathode electrode (7) is 3-5 mm.

3. The electrohydraulic prostate tissue treatment probe, as recited in claim 1, wherein said anode electrode is a platinum-rhodium alloy wire which has a diameter of 0.3 mm.

4. The electrohydraulic prostate tissue treatment probe, as recited in claim 1, wherein said probe pipe body (10) has a wire channel (4) longitudinally provided along the axial direction within an effective length of said probe pipe body (10) and a flexible wire extended between two ends of said wire channel (4) to connect with said probe pipe body to secure into position.

5. The electrohydraulic prostate tissue treatment probe, as recited in claim 1, wherein said cathode electrode (7) is spacedly provided along the axial direction defining a plurality of cathode sections which has an interval of 5 mm, wherein one said cathode section has a length of 5-10 mm and each one of all other said cathode sections has a length of 5 mm.
